(19) United States
(12) Patent Application Publication
(10) Pub. No.: US 2011/0189280 A1
(43) Pub. Date: Aug. 4, 2011

(54) DOSAGE FORM COMPRISING 1-ISOPROPYL-4-HEXAHYDRO-1H-1,4-DIAZEPINE OR A SALT THEREOF

(76) Inventors: Allan James Clarke, Collegeville, PA (US); Yu Li, Collegeville, PA (US); Yinka Bunmi Ogunlana, Essex (GB)

(21) Appl. No.: 13/061,162

(22) PCT Filed: Aug. 21, 2009
(86) PCT No.: PCT/EP2009/060847

§ 371 Date: Apr. 12, 2011

Related U.S. Application Data
(60) Provisional application No. 61/092,911, filed on Aug. 29, 2008.
(60) Provisional application No. 61/234,038, filed on Aug. 14, 2009.

(51) Int. Cl. A61K 9/30; A61K 31/551; A61K 9/36; A61P 25/28; A61P 25/00; A61P 25/18; A61P 25/16

(52) U.S. Cl. 424/465; 424/475; 514/218; 424/480; 427/2.14

(57) ABSTRACT

The invention relates to a dosage form for oral administration comprising a carrier tablet, wherein the carrier tablet is at least partially (preferably partially) covered by a film comprising 1-isopropyl-4-{[4-(tetrahydro-2H-pyran-4-yloxy)phenyl]carbonyl}hexahydro-1H-1,4-diazepine

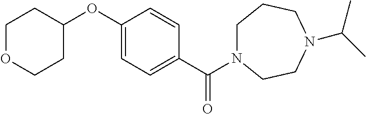

or a pharmaceutically acceptable salt thereof, such as the mono-maleate salt thereof.

In particular embodiments, the film, which at least partially covers the carrier tablet, comprises a stabiliser (e.g. citric acid), and/or a film former (e.g. hydroxypropylcellulose).

The film is preferably present in a recess on the carrier tablet.

The invention also relates to a method of producing said dosage form.

This invention relates to a novel dosage form, to a process for preparing the dosage form and to the use of the dosage form in medicine.

WO 2005/040144 A1 (Glaxo Group Limited) discloses a series of 1-benzoyl-substituted diazepanyl derivatives or a pharmaceutically acceptable salt thereof having affinity for and being antagonists and/or inverse agonists of the histamine H3 receptor, and which are stated therein to be believed to be of potential use in the treatment of neurological diseases including Alzheimer's disease, dementia (including Lewy body dementia and vascular dementia), age-related memory dysfunction, mild cognitive impairment, cognitive deficit, epilepsy, neuropathic pain, inflammatory pain, migraine, Parkinson's disease, multiple sclerosis, stroke and sleep disorders (including narcolepsy and sleep deficits associated with Parkinson's disease); psychiatric disorders including schizophrenia (particularly cognitive deficit of schizophrenia), attention deficit hyperactivity disorder, depression, anxiety and addiction; and other diseases including obesity and gastro-intestinal disorders. WO 2005/040144 A1 discloses this series of compounds or salts thereof for use as a therapeutic substance in the treatment or prophylaxis of the above disorders, in particular cognitive impairments in diseases such as Alzheimer's disease or a related neurodegenerative disorder.

Example 10 of WO 2005/040144 A1 discloses the preparation of 1-(isopropyl)-4-{[4-(tetrahydro-2H-pyran-4-yloxy)phenyl]carbonyl}hexahydro-1H-1,4-diazepine hydrochloride:

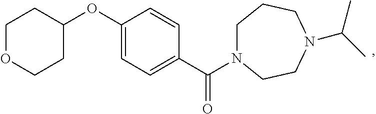

using the following method:

A stirred suspension of 4-(tetrahydro-2H-pyran-4-yloxy)benzoic acid (D6) (222 mg) in dichloromethane (5 ml) at room temperature was treated with oxalyl chloride (0.28 ml) and 10% dimethylformamide in dichloromethane (1 drop). After 1 h the solution was evaporated and then re-evaporated from dichloromethane (2×5 ml). The acid chloride was redissolved in dichloromethane (10 ml) and treated with 1-(isopropyl)-hexahydro-1H-1,4-diazepine dihydrochloride (D2) (178 mg) and diethylaminomethyl polystyrene (3.2 mmol/g, 938 mg). After stirring overnight the mixture was loaded directly on to a silica gel flash column [step gradient 6-10% MeOH (containing 10% 0.880 ammonia solution) in dichloromethane]. Fractions containing the required product were evaporated, then redissolved in dichloromethane and treated with excess 4M HCl in dioxan. Crystallisation from acetone afforded the title compound (E10) (225 mg). MS electrospray (+ion) 347 (MH⁺). ¹H NMR δ (DMSO-d6): 10.45 (1H, m), 7.41 (2H, d, J=8.5 Hz), 7.02 (2H, d, J=8.5 Hz), 4.63 (2H, m), 4.02 (1H, m), 3.02-3.93 (13H, m), 2.32 (1H, m), 1.96 (2H, m), 1.61 (2H, m), 1.27 (6H, d, J=6.5 Hz).

WO 2005/040144 A1 discloses the preparation of the intermediate 4-(tetrahydro-2H-pyran-4-yloxy)benzoic acid (D6, Description 6) as follows:

A solution of ethyl 4-(tetrahydro-2H-pyran-4-yloxy)benzoate (D5) (0.73 g) in ethanol (10 ml) was treated with 1M NaOH (5.84 ml) and the mixture stirred at 60° C. for 5 h. The solution was cooled to room temperature and the ethanol was evaporated. The aqueous was washed with dichloromethane (2×10 ml) and acidified. The solid was filtered off, washed with water and dried to give the title compound (D6) (0.55 g).

WO 2005/040144 A1 discloses the preparation of the intermediate ethyl 4-(tetrahydro-2H-pyran-4-yloxy)benzoate (D5, Description 5) as follows:

An ice-cold solution of ethyl 4-hydroxybenzoate (0.82 g), 4-hydroxy-tetrahydro-2H-pyran (0.5 g) and triphenylphosphine in tetrahydrofuran (50 ml) was treated dropwise with diisopropyl azodicarboxylate (1.69 ml). After 15 min the cooling bath was removed and the reaction stood overnight at room temperature. The mixture was evaporated, redissolved in toluene and successively washed with 2N sodium hydroxide (2×20 ml), water (2×20 ml) and brine (20 ml). After drying (magnesium sulfate) the solution was loaded directly on to a silica flash column (step gradient 10-30% ethyl acetate in light petroleum 40-60) to give the title compound (D5) (0.75 g).

WO 2005/040144 A1 discloses that the 1-benzoyl-substituted diazepanyl derivatives disclosed therein may be included in a pharmaceutical composition, for example a composition adapted for oral, parenteral or rectal administration, which may be in the form of tablets, capsules, oral liquid preparations, powders, granules, lozenges, reconstitutable powders, injectable or infusible solutions or suspensions or suppositories. However, no specific dosage form comprising 1-isopropyl-4-{[4-(tetrahydro-2H-pyran-4-yloxy)phenyl]carbonyl}hexahydro-1H-1,4-diazepine or a pharmaceutically acceptable salt thereof is explicitly disclosed therein.

The present invention involves a dosage form comprising 1-isopropyl-4-{[4-(tetrahydro-2H-pyran-4-yloxy)phenyl]carbonyl}hexahydro-1H-1,4-diazepine or a pharmaceutically acceptable salt thereof, which is suitable for containing reasonably low amounts of the compound or salt, for oral administration. The dosage form of the invention should be able to reduce any potential non-homogeneity of dosing (variation in dose) of the compound or salt, e.g. between different tablets in the same batch, when using low amounts (e.g. ca. 2 mg or less, or ca. 1 mg or less) of the compound or salt.

In a first aspect, the present invention provides a dosage form for oral administration comprising a carrier tablet, wherein the carrier tablet is at least partially (preferably partially) covered by a film comprising 1-isopropyl-4-{[4-(tetrahydro-2H-pyran-4-yloxy)phenyl]carbonyl}hexahydro-1H-1,4-diazepine

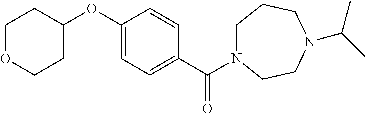

or a pharmaceutically acceptable salt thereof.

In one particular embodiment, the dosage form of the invention comprises:

a) the 1-isopropyl-4-{[4-(tetrahydro-2H-pyran-4-yloxy)phenyl]carbonyl}hexahydro-1H-1,4-diazepine

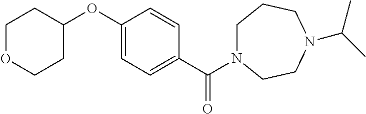

or the pharmaceutically acceptable salt thereof;

b) optionally, a stabiliser, which reduces degradation of the 1-isopropyl-4-{[4-(tetrahydro-2H-pyran-4-yloxy)phenyl]carbonyl}hexahydro-1H-1,4-diazepine or the pharmaceutically acceptable salt thereof in the dosage form when compared to a dosage form lacking said stabiliser; and

c) a pharmaceutically acceptable excipient.

The dosage form is typically adapted for administration to the patient by the oral route of administration. For example, in one particular embodiment, the dosage form adapted for oral administration comprises: a tablet (for example a caplet), a capsule, a pill, or a lozenge; more particularly a tablet.

In the context of this invention, the term “pharmaceutically acceptable excipient” refers to any pharmaceutically acceptable material present in the dosage form other than 1-isopropyl-4-{[4-(tetrahydro-2H-pyran-4-yloxy)phenyl]carbonyl}hexahydro-1H-1,4-diazepine or a pharmaceutically acceptable salt thereof and the stabiliser. Suitable pharmaceutically acceptable excipients will vary depending upon the particular dosage form chosen and include diluents, binders, disintegrants and superdisintegrants, lubricants, glidants, granulating agents, coating agents, wetting agents, solvents, co-solvents, suspending agents, emulsifiers, sweeteners, flavouring agents, flavour-masking agents, colouring agents, anticaking agents, humectants, chelating agents, plasticizers, viscosity increasing agents, rate modifying agents, preservatives, surfactants. The skilled person will appreciate that certain pharmaceutically acceptable excipients may serve more than one function and may serve alternative functions depending on how much of the excipient is present in the formulation and what other ingredients are present in the formulation. Guidance on the selection of suitable pharmaceutically acceptable excipients is available from Remington's Pharmaceutical Sciences (Mack Publishing Company).

The dosage forms of the invention may be prepared using techniques and methods known to those skilled in the art. Some of the methods commonly used in the art are described in Remington's Pharmaceutical Sciences (Mack Publishing Company).

In one particular embodiment, the invention is directed to a solid oral dosage form, such as a tablet or capsule, comprising 1-isopropyl-4-{[4-(tetrahydro-2H-pyran-4-yloxy)phenyl]carbonyl}hexahydro-1H-1,4-diazepine or a pharmaceutically acceptable salt thereof, optionally a stabiliser, and a diluent. Suitably, the diluent includes a saccharide (e.g. lactose, sucrose, or dextrose), a sugar alcohol (e.g. mannitol or sorbitol), starch (e.g. corn starch, potato starch or pregelatinized starch), cellulose or a derivative thereof (e.g. microcrystalline cellulose), calcium sulphate, or dibasic calcium phosphate (CaHPO₄). The dosage form may, in particular, further comprise other excipient(s) such as a binder, a disintegrant, a lubricant and/or a glidant. Suitably, the binder includes starch (e.g. corn starch, potato starch or pregelatinized starch), gelatin, acacia, sodium alginate, alginic acid, tragacanth, guar gum, polyvinylpyrrolidone, or cellulose or a derivative thereof (e.g. ethylcellulose, methylcellulose, carboxymethylcellulose, hydroxypropylcellulose and hydroxypropylmethylcellulose). Suitably, the disintegrant includes a starch, cross-linked polyvinylpyrrolidone, sodium starch glycolate, croscarmellose, alginic acid or sodium carboxymethyl cellulose. Suitably, the lubricant includes stearic acid, magnesium stearate or calcium stearate. Suitably, the glidant includes talc or colloidal silicon dioxide. The oral solid dosage form may further comprise an outer coating which may have cosmetic or functional properties.

In its first aspect, the present invention provides a dosage form for oral administration comprising a carrier tablet, wherein the carrier tablet is at least partially (e.g. partially) covered by a film comprising 1-isopropyl-4-{[4-(tetrahydro-2H-pyran-4-yloxy)phenyl]carbonyl}hexahydro-1H-1,4-diazepine

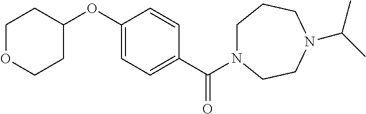

or a pharmaceutically acceptable salt thereof.

Optionally, the film, which at least partially (e.g. partially) covers the carrier tablet, comprises a stabiliser that reduces degradation of the 1-isopropyl-4-{[4-(tetrahydro-2H-pyran-4-yloxy)phenyl]carbonyl}hexahydro-1H-1,4-diazepine or the pharmaceutically acceptable salt thereof in the dosage form, when compared to a dosage form lacking said stabiliser.

In the context of this application, the term “carrier tablet” refers to a tablet that is substantially free of 1-isopropyl-4-{[4-(tetrahydro-2H-pyran-4-yloxy)phenyl]carbonyl}hexahydro-1H-1,4-diazepine or a pharmaceutically acceptable salt thereof. Typically, the tablet does not contain any therapeutic agent, although embodiments in which the carrier tablet contains one or more therapeutic agents are encompassed by the invention.

The composition of the carrier tablet is usually not of the highest importance, provided that it is pharmaceutically acceptable. However, the carrier tablet ideally should be of an appropriate size and shape to function as a tablet for oral administration. Any type of tablet may be used, for example, those described in Remington, The Science and Practice of Pharmacy, 21^{st} Edition, 2005 (Ed. D. B. Troy).

Preferably, the carrier tablet is formed by direct compression technology and/or the carrier tablet comprises a diluent or a mixture of diluents (e.g. present at 60-100% or 70-100%, such as 70-99% or 80-99%, by weight of the carrier tablet). The diluent may include a saccharide (e.g. lactose such as lactose monohydrate or anhydrous lactose, sucrose, or dextrose), a sugar alcohol (e.g. mannitol or sorbitol), starch (e.g. corn starch, potato starch or pregelatinized starch), cellulose (e.g. microcrystalline cellulose, such as Avicel™ PH-102, PH-101, PH-103, PH-112 or PH-113, particularly Avicel™ PH-102), calcium sulphate and/or dibasic calcium phosphate (CaHPO₄).

In a particular embodiment, the diluent comprises microcrystalline cellulose (such as Avicel™ PH-102, PH-101, PH-103, PH-112 or PH-113, particularly Avicel™ PH-102) or lactose (such as lactose monohydrate or anhydrous lactose). In a more particular embodiment, the diluent comprises microcrystalline cellulose (e.g. Avicel™ PH-102). In an alternative particular embodiment, the diluent comprises lactose (such as lactose monohydrate or anhydrous lactose).

The Avicel™ PH-102 and PH-112 grades of microcrystalline cellulose typically have a nominal mean particle size of 100 micrometres. The Avicel™ PH-101, PH-103, and PH-113 grades of microcrystalline cellulose typically have a nominal mean particle size of 50 micrometres. See chapter on “Cellulose, Microcrystalline”, in Handbook of Pharmaceutical Excipients, e.g. 4^{th} edition, 2003, Pharmaceutical Press.

Preferably, the carrier tablet comprises a binder or a mixture of binders, for example present at 1-15%, e.g. 2-12% or 4-12%, by weight of the carrier tablet. The binder can comprise starch (e.g. corn starch, potato starch or pre-gelatinised starch), gelatin, acacia, sodium alginate, alginic acid, tragacanth, guar gum, polyvinylpyrrolidone, or cellulose or a derivative thereof (e.g. ethylcellulose, methylcellulose, carboxymethylcellulose, hydroxypropylcellulose or hydroxypropylmethylcellulose); in particular pregelatinized starch (e.g. Starch 1500).

In one particular embodiment, the carrier tablet contains other excipient(s) such as a lubricant (e.g. stearic acid, magnesium stearate or calcium stearate) and/or a glidant (e.g. talc or colloidal silicon dioxide). In one particular embodiment, a lubricant (in particular magnesium stearate) is present in an amount of from 0.2 to 10%, more particularly 0.2 to 5% or 0.5 to 3%, by weight of the carrier tablet. Alternatively or additionally, in one embodiment, a glidant is present in an amount of from 0.2 to 10%, more particularly 0.2 to 5% or 0.5 to 3%, by weight of the carrier tablet.

In another embodiment, substrates formed by injection moulding such as moulded tablets or capsule shells may be used as carrier tablets. Suitable thermoplastic materials for injection moulding include hydroxypropylcellulose, ethylcellulose, methacrylates and polyvinyl acetate.

In one particular embodiment, the carrier tablet is a tablet comprising microcrystalline cellulose (e.g. Avicel™ PH-102), pregelatinized starch (e.g. Starch 1500) and magnesium stearate. In a more particular embodiment, the carrier tablet has the following composition:

| | % |
|---|---|
| Excipient | w/w |
| Microcrystalline Cellulose (e.g. Avicel ™ PH-102) | 90 |
| Pregelatinized starch (e.g. Starch 1500) | 9 |
| Magnesium Stearate | 1 |

In an alternative embodiment, the carrier substrate may be formulated such that it disintegrates in the mouth when administered orally, a so called “orally disintegrating tablet” or “ODT” substrate. Alternatively, the carrier substrate may be formulated so as to disintegrate (typically rapidly) in water, a so called “fast-dissolve tablet” or “FDT” substrate.

The carrier tablet provides a substrate or support for the film.

In one particular embodiment, the carrier tablets are coated, to substantially prevent absorption of 1-isopropyl-4-{[4-(tetrahydro-2H-pyran-4-yloxy)phenyl]carbonyl}hexahydro-1H-1,4-diazepine or a pharmaceutically acceptable salt thereof by the carrier tablets. However, embodiments in which there is absorption of the 1-isopropyl-4-{[4-(tetrahydro-2H-pyran-4-yloxy)phenyl]carbonyl}hexahydro-1H-1,4-diazepine or a pharmaceutically acceptable salt thereof by the carrier tablets are encompassed by the invention.

Suitable coatings for carrier tablets include aqueous film coats such as those commercially available from Colorcon, for example, an Opadry® coating (e.g. “OPADRY WHITE 00F18484”™ or “OPADRY WHITE YS-1-7003”™). Other suitable coatings include Surelease® (ethylcellulose). The dosage form may alternatively be coated with a film of gastroresistant and enterosoluble polymeric material. Suitable polymeric materials include cellulose acetophthalate, cellulose acetopropionate, cellulose trimellitate and acrylic and methacrylic copolymers. Colourings can be added to the coating.

In one embodiment, the carrier tablet is coated with a film coat to a 2-6% weight gain.

It will be appreciated that, in embodiments where the carrier tablet is coated to substantially prevent absorption of 1-isopropyl-4-{[4-(tetrahydro-2H-pyran-4-yloxy)phenyl]carbonyl}hexahydro-1H-1,4-diazepine or a pharmaceutically acceptable salt thereof, the film coat selected must not be soluble in the solvent used during the manufacturing process of the dosage form. For example, where an aqueous solvent system is used, an aqueous film coat (like an Opadry®) will immediately disintegrate and a coating not soluble in water (e.g. Surelease® or Eudragit®) is appropriate.

In one particular embodiment, the film containing the 1-isopropyl-4-{[4-(tetrahydro-2H-pyran-4-yloxy)phenyl]carbonyl}hexahydro-1H-1,4-diazepine or the pharmaceutically acceptable salt thereof only partially coats the carrier tablet. In a more particular embodiment, the carrier tablets are shaped to contain one or more recesses or depressions. In such embodiments, the film containing 1-isopropyl-4-{[4-(tetrahydro-2H-pyran-4-yloxy)phenyl]carbonyl}hexahydro-1H-1,4-diazepine or a pharmaceutically acceptable salt thereof may be substantially present within the recess of the carrier tablet.

The dosage form, and/or the film that at least partially covers the carrier tablet, comprises 1-isopropyl-4-{[4-(tetrahydro-2H-pyran-4-yloxy)phenyl]carbonyl}hexahydro-1H-1,4-diazepine (the “free base”) or a pharmaceutically acceptable salt thereof. In the context of this invention, reference to the free base or pharmaceutically acceptable salt encompasses solvates and hydrates of the free base or pharmaceutically acceptable salt.

1-Isopropyl-4-{[4-(tetrahydro-2H-pyran-4-yloxy)phenyl]carbonyl}hexahydro-1H-1,4-diazepine or a pharmaceutically acceptable salt thereof, such as the hydrochloride salt thereof or more preferably the mono-maleate salt thereof, may be prepared by procedures such as those disclosed in WO 2005/040144 A1. The relevant preparation procedures disclosed in WO 2005/040144 A1 are incorporated herein by reference; some of these (Example 10 and Descriptions 6 and 5 of WO 2005/040144 A1) are also mentioned hereinabove. The intermediate 1-(isopropyl)-hexahydro-1H-1,4-diazepine dihydrochloride (D2) and its precursor 1-tert-butyl-4-(isopropyl)-hexahydro-1H-1,4-diazepine-1-carboxylate can be prepared as described in Descriptions 2 and 1 respectively of WO 2005/040144 A1.

The pharmaceutically acceptable salt of 1-isopropyl-4-{[4-(tetrahydro-2H-pyran-4-yloxy)phenyl]carbonyl}hexahydro-1H-1,4-diazepine can be a pharmaceutically acceptable acid addition salt, such as the hydrochloride salt or more preferably the mono-maleate salt thereof (such as crystalline Form 1 of the mono-maleate salt thereof). Such salts can be formed by reaction (mixture) with the appropriate acid (e.g. maleic acid or HCl), typically in a suitable solvent such as an organic solvent (e.g. ethyl acetate as solvent when preparing the mono-maleate salt), to give the salt which can be isolated for example by crystallisation and filtration (see e.g. Drug Preparation Examples 1, 2 and 3 hereinafter).

The Drug Preparation Examples 1, 2 and 3 hereinafter also describe particular preparations of the “free base” compound 1-isopropyl-4-{[4-(tetrahydro-2H-pyran-4-yloxy)phenyl]carbonyl}hexahydro-1H-1,4-diazepine, as well as particular preparations of the hydrochloride and mono-maleate salts thereof.

In the dosage form of the present invention, the dosage form and/or the film may contain the free base, a pharmaceutically acceptable salt (stoichiometric or non-stoichiometric), or any mixture of these.

Preferably, the dosage form contains 1-isopropyl-4-{[4-(tetrahydro-2H-pyran-4-yloxy)phenyl]carbonyl}hexahydro-1H-1,4-diazepine mono-maleate (e.g. crystalline Form 1 thereof). In one particular embodiment, the film contains 1-isopropyl-4-{[4-(tetrahydro-2H-pyran-4-yloxy)phenyl]carbonyl}hexahydro-1H-1,4-diazepine mono-maleate (e.g. crystalline Form 1 thereof).

In a particular embodiment, crystalline Form 1 of 1-isopropyl-4-{[4-(tetrahydro-2H-pyran-4-yloxy)phenyl]carbonyl}hexahydro-1H-1,4-diazepine mono-maleate has an X-ray powder diffraction (XRPD) diffractogram comprising four or more (or preferably five or more, more preferably six or more, or most preferably all) of the following peaks at substantially the following degrees two-theta (2θ) values:

9.2±0.1°, 13.4±0.1°, 17.0±0.1°, 18.5±0.1°, 19.8±0.1°, 21.3±0.1°, and 27.8±0.1°;

wherein the X-ray powder diffraction diffractogram is measured with a X-ray powder diffractometer using copper K-alpha X-radiation and a step size of 0.0167° two-theta or less. The XRPD diffractogram is preferably measured using a time per step of 31.75 seconds or more, and/or using a start angle of 2° two-theta (2θ) and an end angle of 40° two-theta (2θ); and/or is preferably measured using a sample mounted on a silicon wafer plate (typically a silicon wafer zero background plate), and/or using a sample which is a layer (e.g. a thin layer) of powder.

In a particular embodiment, crystalline Form 1 of 1-isopropyl-4-{[4-(tetrahydro-2H-pyran-4-yloxy)phenyl]carbonyl}hexahydro-1H-1,4-diazepine mono-maleate has a solid-form attenuated total reflectance (ATR) infrared (IR) spectrum comprising five or more (or preferably six or more, more preferably seven or more, still more preferably eight or more, yet more preferably ten or more, or most preferably all) of the following peaks:

1700, 1622, 1464, 1422, 1353, 1247, 1234, 1089, 1048, 869, 840 and 765 cm⁻¹;

with a variation allowed for each peak of ±2 cm⁻¹. The solid-form IR spectrum is preferably measured using an FT-IR (Fourier Transform Infrared) spectrometer, such as an FT-IR spectrometer fitted with an attenuated total reflectance (ATR) sampling accessory (e.g. a diamond/ZnSe ATR sampling accessory), and/or is measured at 4 cm⁻¹ resolution. In any IR spectrum plotting percent transmittance on the vertical axis with high transmittance at the top of the spectrum, a vibrational mode or band causing an absorption of IR radiation will be shown as a down-pointing trough or valley (of lower transmittance) and not as an up-pointing peak. Therefore, the term “peak” or “band”, when used herein in respect of an IR spectrum, includes a down-pointing trough representing an absorption or decreased transmittance of IR radiation.

Gravimetric vapour sorption (GVS) analysis, which measures water sorption/desorption at different relative humidities, shows that crystalline Form 1 of 1-isopropyl-4-{[4-(tetrahydro-2H-pyran-4-yloxy)phenyl]carbonyl}hexahydro-1H-1,4-diazepine mono-maleate, reversibly adsorbs and/or absorbs, and/or desorbs, approximately 0.4% w/w water across the 0-90% relative humidity range at 25° C. under nitrogen. This represents an advantage, in respect of drug developability, over the “kinetic solid” 1-isopropyl-4-{[4-(tetrahydro-2H-pyran-4-yloxy)phenyl]carbonyl}hexahydro-1H-1,4-diazepine hydrochloride prepared in Drug Preparation Example 2 Part C (the sample before purification by recrystallisation), which adsorbs and/or absorbs, and/or desorbs, higher quantities of water across the 0-90% relative humidity range at 25° C. under nitrogen.

In one particular embodiment of the invention, the dosage form and/or the film contains from 10 μg to 2 mg or 20 μg to 2 mg of the 1-isopropyl-4-{[4-(tetrahydro-2H-pyran-4-yloxy)phenyl]carbonyl}hexahydro-1H-1,4-diazepine or the pharmaceutically acceptable salt thereof, when measured as the amount of base present (that is, excluding any amount of acid added to form any salt). In a more particular embodiment, the dosage form and/or the film contains from 20 μg to 1 mg, particularly from 50 μg to 1 mg, more particularly from 50 μg to 500 μg, of the 1-isopropyl-4-{[4-(tetrahydro-2H-pyran-4-yloxy)phenyl]carbonyl}hexahydro-1H-1,4-diazepine or the pharmaceutically acceptable salt thereof, when measured as the amount of free base present.

The dosage form, e.g. containing the above-mentioned amounts of the compound or salt, may be administered once per day or more than once a day, for example two or three times a day, in particular by oral administration e.g. to a human.

The dosage form may be of potential use in the treatment of neurological diseases including cognitive impairment, Alzheimer's disease, dementia (e.g. Lewy body dementia or vascular dementia), age-related memory dysfunction, mild cognitive impairment, epilepsy, neuropathic pain, Parkinson's disease, multiple sclerosis, stroke, or a sleep disorder (such as narcolepsy or sleep deficits associated with Parkinson's disease); or a psychiatric disorder such as schizophrenia (particularly cognitive impairment in schizophrenia), attention deficit hyperactivity disorder, depression, anxiety or addiction; e.g. in a mammal such as a human.

The dosage form may be for use in the treatment or prophylaxis (e.g. treatment) of any of the above disorders, in particular (a) cognitive impairment, e.g cognitive impairment in a disease such as Alzheimer's disease, dementia (e.g. Lewy body dementia or vascular dementia), age-related memory dysfunction, mild cognitive impairment, or a related neurodegenerative disorder, or cognitive impairment in schizophrenia; or (b) a sleep disorder (such as narcolepsy or sleep deficits associated with Parkinson's disease); e.g. in a mammal such as a human.

The invention further provides the dosage form as defined herein for use in the treatment of a neurological disease in a mammal such as a human.

The invention further provides a method of treatment or prophylaxis of any of the above disorders, in mammals including humans, which comprises administering to the sufferer the dosage form of the present invention.

The invention further provides a method of treatment of a neurological disease which comprises administering to a mammalian (e.g. human) host in need thereof a dosage form of the present invention as defined herein.

In another aspect, the invention provides the use of 1-isopropyl-4-{[4-(tetrahydro-2H-pyran-4-yloxy)phenyl]carbonyl}hexahydro-1H-1,4-diazepine or a pharmaceutically acceptable salt thereof in the manufacture of the dosage form of the invention for use in the treatment of any of the above disorders, e.g. in a mammal such as a human.

The invention further provides the use of 1-isopropyl-4-{[4-(tetrahydro-2H-pyran-4-yloxy)phenyl]carbonyl}hexahydro-1H-1,4-diazepine or a pharmaceutically acceptable salt thereof in the manufacture of a dosage form of the present invention as defined herein, for the treatment of a neurological disease in a mammal such as a human.

The dosage form and/or the film additionally optionally contains a pharmaceutically acceptable stabiliser that reduces degradation of 1-isopropyl-4-{[4-(tetrahydro-2H-pyran-4-yloxy)phenyl]carbonyl}hexahydro-1H-1,4-diazepine or a pharmaceutically acceptable salt thereof in the dosage form containing the stabiliser when compared to a dosage form lacking the stabiliser. The degradation of 1-isopropyl-4-{[4-(tetrahydro-2H-pyran-4-yloxy)phenyl]carbonyl}hexahydro-1H-1,4-diazepine or a pharmaceutically acceptable salt thereof in the dosage form can be analysed by measuring the total impurity/degradation product content of the dosage form using gradient HPLC, using the method described below. The skilled reader will appreciate that, with the exception of the presence or absence of stabiliser, the dosage forms should otherwise be comparable, and should have been stored under similar conditions for similar time periods.

In one embodiment, the mean total impurity/degradation product content calculated from at least 3 samples of the dosage form containing stabiliser stored for 1 month at 40° C., 75% relative humidity is at least 50% lower than the mean total impurity/degradation product content calculated from at least 3 samples of a comparable dosage form lacking said stabiliser that was stored under comparable conditions.

In another embodiment, the mean total impurity/degradation product content of at least 3 samples of the dosage form containing stabiliser when stored at 30° C., 65% relative humidity for a period of 3 months should not exceed 10%, more particularly 5%.

Certain pharmaceutically acceptable antioxidants may act as stabilisers in the context of the present invention. Pharmaceutically acceptable antioxidants include those described in The Handbook of Pharmaceutical Excipients, Third Edition, 2000 (Ed. A. H. Kibbe).

In one particular embodiment, the stabiliser comprises a pharmaceutically acceptable antioxidant, and/or the stabiliser comprises a pharmaceutically acceptable organic acid. In one particular embodiment, the stabiliser comprises a pharmaceutically acceptable antioxidant which is an organic acid.

In one particular embodiment, the stabiliser is selected from the group consisting of citric acid, a salt of citric acid (e.g. sodium citrate, e.g. mono-, di-, or tri-sodium citrate), malic acid, a salt of malic acid, maleic acid, a salt of maleic acid, ascorbic acid, a salt of ascorbic acid (e.g. sodium ascorbate), tartaric acid, a salt of tartaric acid, and combinations thereof.

In one particular embodiment, the stabiliser is selected from the group consisting of citric acid, malic acid, ascorbic acid, a salt of ascorbic acid, sodium bicarbonate, butylated hydroxyanisole and butylated hydroxytoluene. Combinations of stabilisers may also be used in the present invention.

More particularly, the stabiliser is selected from the group consisting of citric acid, malic acid, ascorbic acid, a salt of ascorbic acid, and combinations thereof.

Still more particularly, the stabiliser is selected from the group consisting of citric acid, malic acid and ascorbic acid.

In one yet more particular embodiment, the stabiliser comprises (and/or the film contains) citric acid.

In an alternative embodiment, the stabiliser comprises (and/or the film contains) maleic acid (e.g. when the active compound or salt is the mono-maleate thereof).

The optional stabiliser or stabilisers, if present, are ideally present in the dosage form and/or the film in a sufficient amount to reduce degradation of 1-isopropyl-4-{[4-(tetrahydro-2H-pyran-4-yloxy)phenyl]carbonyl}hexahydro-1H-1,4-diazepine or a pharmaceutically acceptable salt thereof. In certain particular embodiments comprising citric acid, the molar ratio of the 1-isopropyl-4-{[4-(tetrahydro-2H-pyran-4-yloxy)phenyl]carbonyl}hexahydro-1H-1,4-diazepine or the pharmaceutically acceptable salt thereof (measured as the free base) to the citric acid is in the range of 1.5:1 to 1:500.

In one particular embodiment, the stabiliser (e.g. citric acid) is present in 10% to 65%, or 20% to 50%, or 25% to 45% (e.g. ca. 33% or ca. 37.5%) by weight of the film; and/or is present in 0.005% to 2% (e.g. 0.01% to 1%, or 0.01% to 0.5%, or 0.03% to 0.2%, e.g. ca. 0.08%) by weight of the dosage form (e.g. tablet).

Preferably, the film, which at least partially (e.g. partially) covers the carrier tablet, additionally contains a film former, such as hydroxypropylcellulose (HPC), hydroxypropylmethyl cellulose (HPMC), hydroxyethylcellulose (HEC), carboxymethyl cellulose, polyvinyl alcohol, polyvinyl pyrrolidone, carrageenan (e.g. kappa iota or lambda), gelatin, polyethylene glycol, polyethylene oxide, pullulan, or a methacrylic acid polymer (e.g. EUDRAGIT grades RL, RS, E, L, S, FS30D), or any combination thereof.

More preferably, the film, which at least partially covers the carrier tablet, contains a film former which is hydroxypropylcellulose (HPC). The film former can for example be HPC of the grade Klucel™ Grade EF (available from Aqualon, 1313 North Market St, Wilmington, Del. 19894-0001, US). HPC of Klucel™ Grade EF has a weight averaged molecular weight of 80000, and a typical Brookfield viscosity of 300-600 mPa·s when present at 10% concentration in aqueous solution (based on Aqualon Pharmaceutical Excipients catalogue). Preferably, however, the film former is Nisso™ HPC (hydroxypropylcellulose) Grade SSL, Nisso™ HPC Grade SL, or Nisso™ HPC Grade L (each grade having a different molecular weight), available from Nisso America Inc., 45 Broadway, Suite 2120, New York, N.Y. 10006, USA (or from Nippon Soda Co., Ltd., Japan). Typically, Nisso™ HPC (hydroxypropylcellulose) Grades SSL, SL, and L have average molecular weights of: 34000, 77000, and 125000 respectively (source: Nippon Soda Co., Ltd.). Nisso™ HPC (hydroxypropylcellulose) Grades SSL, SL, and L respectively have viscosities of: 2.0 to 2.9 (typically 2.5) mPa·s, 3.0 to 5.9 (typically 4.7) mPa·s, and 6.0 to 10.0 (typically 7.9) mPa·s, when measured as aqueous solutions containing 2% by weight of dry HPC at 20° C. (sources: Nisso America Inc. and Nippon Soda Co., Ltd.).

Preferably therefore, the film, which at least partially (e.g. partially) covers the carrier tablet, contains a film former which is hydroxypropylcellulose (HPC), and wherein: (a) the HPC has an average molecular weight of from 34000 to 125000 (e.g. 34000, 77000, 80000, or 125000); and/or (b) the HPC has a viscosity in aqueous solution of from 2.0 to 10.0 mPa·s [e.g. from 2.0 to 2.9 (e.g. 2.5) mPa·s, or from 3.0 to 5.9 (e.g. 4.7) mPa·s, or from 6.0 to 10.0 (e.g. 7.9) mPa·s], when measured as an aqueous solution containing 2% by weight of dry HPC at 20° C. (e.g. using a Brookfield rheometer).

Typically, any film former included in the dosage form is soluble in the solvent used during its production.

In one particular embodiment, the film former (e.g. hydroxypropylcellulose) is present in 30% to 99.5%, or 30% to 95%, or 40% to 95%, or 50% to 90%, or 50% to 80% (e.g. ca. 66-67% or ca. 62.5%) by weight of the film; and/or is present in 0.01% to 4% (e.g. 0.02% to 2%, or 0.02% to 1%, or 0.05% to 0.4%, e.g. ca. 0.13%) by weight of the dosage form (e.g. tablet).

The film may additionally contain other excipients. For example, it has been found that certain solvent systems, such as aqueous systems, require addition of surfactants (e.g. polysorbates (20, 40, 80), Triton 100, sodium lauryl sulphate or tyloxopol) and/or antifoaming agents (polydimethylsiloxane or dimethicone). Therefore, in a further embodiment, the film additionally contains one or more surfactants and/or one or more antifoaming agents.

The dosage form may be further coated (i.e. may comprise a further coating). Preferably, the further coating comprises titanium dioxide and/or hydroxypropylcellulose (HPC) such as Nisso™ HPC Grade SSL; more preferably, the further coating comprises 25-55% HPC (e.g. Nisso™ HPC Grade SSL) and 45-75% titanium dioxide, by weight of the dried further coating. More preferably the further coating process uses a mixture of HPC and/or titanium dioxide in ethanol (e.g. using a pad printing overcoat process—e.g. see description hereinafter). Still more preferably, the further coating process (e.g. pad printing process) uses a coating mixture having the following generalised composition: 20 to 25% Nisso™ HPC SSL; 31 to 38% titanium dioxide; and 41 to 49% ethanol.

Other possibly suitable further coatings include those listed above as suitable for coating of the carrier tablet. Therefore, suitably, the further coating comprises an aqueous film coat such as one commercially available from Colorcon, for example, an Opadry® coating (e.g. “OPADRY WHITE 00F18484”™ or “OPADRY WHITE YS-1-7003”™). Other suitable further coatings include Surelease® (ethylcellulose). The dosage form may alternatively be further coated with a film of gastroresistant and enterosoluble polymeric material. Suitable polymeric materials include cellulose acetophthalate, cellulose acetopropionate, cellulose trimellitate and acrylic and methacrylic copolymers.

Colourings can optionally be added to the further coating.

In one particular embodiment, the dosage form is further coated to 2-6% weight gain.

In a particular embodiment, the further coating forms a coating over the film (more particularly a coating over a majority or all of the film, preferably a coating over all of the film) which contains the 1-isopropyl-4-{[4-(tetrahydro-2H-pyran-4-yloxy)phenyl]carbonyl}hexahydro-1H-1,4-diazepine or the pharmaceutically acceptable salt thereof.

In a particular embodiment, the further coating is a pad printed overcoat, i.e. is an overcoat (a coating over a majority or all (preferably all) of the film which contains the 1-isopropyl-4-{[4-(tetrahydro-2H-pyran-4-yloxy)phenyl]carbonyl}hexahydro-1H-1,4-diazepine or the pharmaceutically acceptable salt thereof) which is formed by a pad printing process.

The dosage form may optionally be packaged in a low oxygen environment. This may be achieved by inclusion of an oxygen scavenger in the packaging of the dosage form. Suitable oxygen scavengers include PharmaKeep® KH and KD (commercially available from Sud Chemie) and StabilOx™ speciality oxygen scavenger (commercially available from Multisorb Technologies). Alternatively, the dosage forms can be packaged in bottles that are impermeable to oxygen. Aluminium-aluminium blisters may also be used to package the dosage forms in a low oxygen environment.

In another aspect, the invention provides a method for preparing the dosage form of the invention. The method comprises dispensing a solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, onto a carrier tablet. Any solvent may be used provided that the optional stabiliser and any other excipients present in the film are soluble in the solvent. The solvent is typically volatile, and must be pharmaceutically acceptable in the (residual) quantities in which it appears in the finished dosage form.

Suitably, the solvent includes water, organic solvent(s), propellants, liquefied gases or volatile silicone(s). In one particular embodiment, the solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and an optional stabiliser, is prepared using an organic solvent, such as methanol, ethanol, acetone, acetic acid, or methylene chloride(dichloromethane). Mixtures of solvents (e.g. water-ethanol) may also be used.

In a particular embodiment, the solvent is an organic solvent or a mixture of solvents (e.g. an organic solvent or a mixture of organic solvents). In a particular embodiment, the organic solvent is methanol, ethanol, 1-propanol, isopropanol, 1-butanol, isobutanol, acetone, methyl ethyl ketone, tetrahydrofuran, ethyl acetate, isopropyl acetate, methyl acetate, acetic acid, methylene chloride(dichloromethane), or cyclohexane. More particularly, the organic solvent is methanol, ethanol, acetone, acetic acid or methylene chloride(dichloromethane). In one preferred embodiment, the solvent is methanol.

In a particular embodiment, in the method for preparing the dosage form, the solution or suspension of the 1-isopropyl-4-{[4-(tetrahydro-2H-pyran-4-yloxy)phenyl]carbonyl}hexahydro-1H-1,4-diazepine or the pharmaceutically acceptable salt thereof is prepared using, as a starting material, solid (e.g. crystalline) 1-isopropyl-4-{[4-(tetrahydro-2H-pyran-4-yloxy)phenyl]carbonyl}hexahydro-1H-1,4-diazepine mono-maleate; or more particularly using, as a starting material, crystalline Form 1 of 1-isopropyl-4-{[4-(tetrahydro-2H-pyran-4-yloxy)phenyl]carbonyl}hexahydro-1H-1,4-diazepine mono-maleate e.g. characterized by having:

(a) an X-ray powder diffraction diffractogram comprising four or more of the following peaks at substantially the following degrees two-theta values:

9.2±0.1°, 13.4±0.1°, 17.0±0.1°, 18.5±0.1°, 19.8±0.1°, 21.3±0.1°, and 27.8±0.1°;

wherein the X-ray powder diffraction diffractogram is measured with a X-ray powder diffractometer using copper K-alpha X-radiation and a step size of 0.0167° two-theta or less; and/or

(b) a solid-form attenuated total reflectance infrared spectrum comprising five or more of the following peaks:

1700, 1622, 1464, 1422, 1353, 1247, 1234, 1089, 1048, 869, 840 and 765 cm⁻¹;

with a variation allowed for each peak of ±2 cm⁻¹.

In a further aspect, the invention provides a solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and an optional stabiliser, in a solvent system. In one embodiment, the solution or suspension further comprises one or more film formers and/or surfactants and/or antifoaming agents. In another embodiment, the solvent is an organic solvent, such as methanol, ethanol, acetone, acetic acid or methylene chloride, more particularly methanol.

In certain particular embodiments in which the stabiliser is citric acid, it is present in the solution or suspension in an amount between 2-3% w/v, particularly 3% w/v.

In certain embodiments in which the stabiliser is butylated hydroxyanisole, it is present in the solution or suspension in an amount between 0.01-0.1% w/v, particularly 0.02% w/v.

In certain particular embodiments in which the film former is hydroxypropylcellulose (HPC) (e.g. HPC as further defined above), it is present in the solution or suspension in an amount between 4-6% w/v, particularly 4% w/v or 5% w/v.

The carrier tablet and dispensed solution/suspension may be heated (e.g. in a forced air oven, e.g. at 40-60° C. such as about 50° C., and/or e.g. for about 10-20 minutes) to evaporate excessive liquid and result in the formation of a film upon at least a part of the surface of the carrier tablet. The dosage form may then optionally be film coated, e.g. according to methods known in the art and/or as described herein.

The carrier tablet used in the method for preparing the dosage form may have a recess or depression that provides a basin for the solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and an optional stabiliser, to land after being dispensed. Typically, biconcave tablets having recesses on two faces of the tablet are employed. The two recesses can be used to receive the solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser. Alternatively, one of the recesses can be used to receive a solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, and the remaining recess can be used to receive a solution or suspension of another therapeutic agent to produce a dosage form containing two different therapeutic agents. In a further embodiment, solutions of different therapeutic agents may be layered one on top of the other.

The dosage form of the present invention may be produced using the apparatus described in WO2005/123569 which publication is herein incorporated in its entirety. More particularly, the dosage form of the present invention may be produced by an apparatus containing a dispensing module for accurately dispensing a predetermined amount of the solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, onto the carrier tablets. The apparatus may also have a holding member for holding the carrier tablets, which may move continually along the apparatus as the dispensing module dispenses the solution/suspension onto each of the carrier tablets.

The apparatus may also have a drying system that dries or evaporates solvent from the solution/suspension deposited on each of the carrier tablets. The holding member may move continually along the apparatus as the drying system dries the dosage on each of the carrier tablets. The drying system may dry the dosage by use of heated air, infrared or microwave heating.

The apparatus may also have a coating system that applies a coating over the dosage form. The coating system may have a pad printing device or a sprayer that applies a or the coating to each of the carrier substrates, or to the dosage form comprising the 1-isopropyl-4-{[4-(tetrahydro-2H-pyran-4-yloxy)phenyl]carbonyl}hexahydro-1H-1,4-diazepine or the pharmaceutically acceptable salt thereof (e.g. a pad printing device or sprayer that applies a or the coating to the film comprising the 1-isopropyl-4-{[4-(tetrahydro-2H-pyran-4-yloxy)phenyl]carbonyl}hexahydro-1H-1,4-diazepine or the pharmaceutically acceptable salt thereof). The holding member may move continually along the apparatus as the coating system applies the coating to each of the carrier tablets. The apparatus may also have a coating dryer that dries the coating on each of the carrier tablets.

It will be apparent that the apparatus described above could re-process carrier tablets any number of times in order to add solutions or suspensions of different therapeutic agents. Alternatively, the apparatus could have additional dispensing systems in series to add each of the solutions/suspensions to the carrier tablets.

1-isopropyl-4-{[4-(tetrahydro-2H-pyran-4-yloxy)phenyl]carbonyl}hexahydro-1H-1,4-diazepine or a pharmaceutically acceptable salt thereof may be used in combination with other therapeutic agents. When 1-isopropyl-4-{[4-(tetrahydro-2H-pyran-4-yloxy)phenyl]carbonyl}hexahydro-1H-1,4-diazepine or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride and rivastigmine), nicotinic receptor agonists or allosteric modulators (such as α7 agonists or allosteric modulators or α4β2 agonists or allosteric modulators), PPAR agonists (such as PPARγ agonists), 5-HT₄ receptor partial agonists, 5-HT₆ receptor antagonists or 5HT1A receptor antagonists and NMDA receptor antagonists or modulators, or disease modifying agents such as β or γ-secretase inhibitors.

When 1-isopropyl-4-{[4-(tetrahydro-2H-pyran-4-yloxy)phenyl]carbonyl}hexahydro-1H-1,4-diazepine or a pharmaceutically acceptable salt thereof is intended for use in the treatment of narcolepsy, it may be used in combination with medicaments claimed to be useful as treatments for narcolepsy. Suitable examples of such other therapeutic agents include modafinil, armodafinil and monoamine uptake blockers.

When 1-isopropyl-4-{[4-(tetrahydro-2H-pyran-4-yloxy)phenyl]carbonyl}hexahydro-1H-1,4-diazepine or a pharmaceutically acceptable salt thereof is intended for use in the treatment of schizophrenia, it may be used in combination with medicaments claimed to be useful as treatments of schizophrenia including i) antipsychotics including typical antipsychotics (for example chlorpromazine, thioridazine, mesoridazine, fluphenazine, perphenazine, prochlorperazine, trifluoperazine, thiothixine, haloperidol, molindone and loxapine), atypical antipsychotics (for example clozapine, olanzapine, risperidone, quetiapine, aripirazole, ziprasidone, amisulpride and aripiprazole), glycine transporter 1 inhibitors and metabotropic receptor ligands; ii) drugs for extrapyramidal side effects, for example anticholinergics (such as benztropine, biperiden, procyclidine, and trihexyphenidyl) and dopaminergics (such as amantadine); iii) antidepressants including serotonin reuptake inhibitors (such as citalopram, escitalopram, fluoxetine, paroxetine, dapoxetine and sertraline), dual serotonin/noradrenaline reuptake inhibitors (such as venlafaxine, duloxetine and milnacipran), noradrenaline reuptake inhibitors (such as reboxetine), tricyclic antidepressants (such as amitriptyline, clomipramine, imipramine, maprotiline, nortriptyline and trimipramine), monoamine oxidase inhibitors (such as isocarboxazide, moclobemide, phenelzine and tranylcypromine), and others (such as buproprion, mianserin, mirtazepine, nefazodone and trazodone); iv) anxiolytics including benzodiazepines such as alprazolam and lorazepam; and v) cognitive enhancers for example cholinesterase inhibitors (such as tacrine, donepezil, rivastigmine and galantamine).

The invention thus provides, in a further aspect, a dosage form comprising a carrier tablet which carrier tablet is at least partially coated by a film comprising 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, which dosage form further comprises an additional therapeutic agent or agents.

It will be apparent that the additional therapeutic agent may be present in the carrier tablet. Alternatively, as discussed above, a film containing additional therapeutic agents may be deposited on the carrier tablet. Where the carrier tablet has two recesses, one recess may contain the film containing 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, and the second recess may contain the film containing the additional therapeutic agent or agents. Alternatively, the films containing 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, and the additional therapeutic agent may be layered one on top of the other.

When 1-isopropyl-4-{[4-(tetrahydro-2H-pyran-4-yloxy)phenyl]carbonyl}hexahydro-1H-1,4-diazepine or a pharmaceutically acceptable salt thereof is used in combination with a second therapeutic agent active against the same disease state the dose of 1-isopropyl-4-{[4-(tetrahydro-2H-pyran-4-yloxy)phenyl]carbonyl}hexahydro-1H-1,4-diazepine or a pharmaceutically acceptable salt thereof may differ from that when the 1-isopropyl-4-{[4-(tetrahydro-2H-pyran-4-yloxy)phenyl]carbonyl}hexahydro-1H-1,4-diazepine or a pharmaceutically acceptable salt thereof is formulated alone. Appropriate doses will be readily appreciated by those skilled in the art.

The following examples illustrate the invention but do not limit it in any way.

### DRUG PREPARATION EXAMPLES

The following Drug Preparation Examples describe particular preparations of the “free base” compound 1-isopropyl-4-{[4-(tetrahydro-2H-pyran-4-yloxy)phenyl]carbonyl}hexahydro-1H-1,4-diazepine, as well as of the hydrochloride and mono-maleate salts thereof.

Abbreviations, some of which are used herein, include:

DMSO dimethyl sulfoxide

eq equivalents

HPLC high performance liquid chromatography

h hour(s)

min minute(s)

GC gas chromatography

LCMS or LC/MS liquid chromatography/mass spectrometry

NMR nuclear magnetic resonance

¹H NMR ¹H nuclear magnetic resonance, in which br=broad, m=multiplet, s=singlet, d=doublet, t=triplet, td=triplet of doublets, etc, and 1H or 2H=integral shows one hydrogen or two hydrogens, etc.

TLC thin layer chromatography

Room temperature (ambient temperature): This is usually in the range of about 17 to about 25° C., or a sub-range within this range, except as disclosed herein.

Drug Preparation Intermediate 1

### 1-Isopropyl-hexahydro-1H-1,4-diazepine bis-trifluoroacetate salt

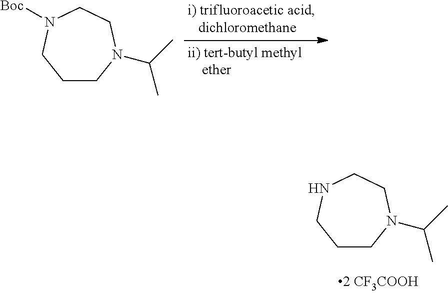

Process Summary

All weights, volumes and equivalents are relative to 1-tert-butyl-4-isopropyl-hexahydro-1H-1,4-diazepine-1-carboxylate.

A solution of 1-tert-butyl-4-isopropyl-hexahydro-1H-1,4-diazepine-1-carboxylate (1 weight, e.g. which can optionally be prepared as described in Description 1 Method A or B on page 10 of WO 2005/040144 A1) in dichloromethane (14 volumes) is cooled to 0±3° C. Trifluoroacetic acid (5 weight, 3.4 volumes, 10 mole equivalents) is added over at least 30 min. The reaction mixture is then warmed to 30±3° C. and stirred at this temperature for at least 12 hours. Once the reaction is complete by TLC and GC, the mixture is evaporated to dryness on a rotary evaporator. Ethyl acetate (7 volumes) is added to the residue and the mixture is evaporated to dryness. This is repeated once. Ethyl acetate (0.5 volumes) is added to the resulting oil followed by tert-butyl methyl ether (10 volumes) and the mixture is stirred for 2 hours. The resulting solid is filtered off under vacuum and is washed with tert-butyl methyl ether (2×3 volumes). The solid is then dried in vacuo at 40° C. until a constant probe temperature is achieved, to give solid 1-isopropyl-hexahydro-1H-1,4-diazepine bis-trifluoroacetate salt.

Drug Preparation Intermediate 2

### 4-(Tetrahydro-2H-pyran-4-yloxy)benzoic acid

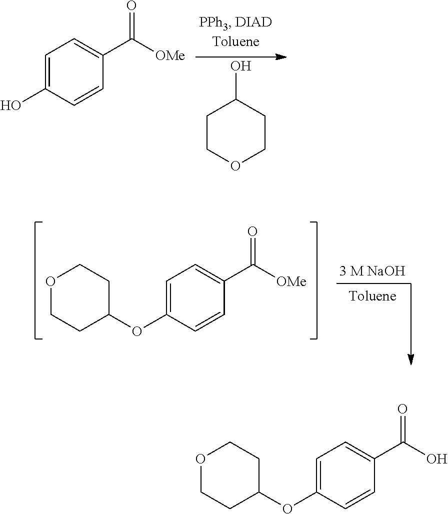

Short Summary Process Description

All weights, volumes (“vol”) and equivalents are relative to methyl 4-hydroxybenzoate.

A solution of methyl 4-hydroxybenzoate (1 wt, 1 mole equivalent), triphenyl phosphine (2.6 wt, 1.5 mole equivalents), 4-hydroxytetrahydropyran (0.75 vol, 1.2 mole equivalents) in toluene (3.5 vol) under nitrogen is heated to 55° C. and diisopropyl azodicarboxylate (1.95 vol, 1.5 mole equivalents) is added dropwise over 60 minutes, maintaining the contents at 60±2° C. Following the addition, the reaction is stirred for 30 minutes, and then cooled to 0-5° C. The batch is then seeded with pre-prepared triphenylphosphine oxide-diisopropyl hydrazinedicarboxylate adduct, and then allowed to stir for a further 1 hour before filtering. The wet cake is washed with toluene (2×1 vol), and the combined mother liquors are transferred into a clean vessel. The toluene solution is washed with 2M sodium hydroxide solution (5 vol) at 0-5° C., and then 3M sodium hydroxide solution (5 vol) is added and the reaction is heated to 80° C. The reaction is stirred for at least 2.5 hours, until HPLC shows no starting material. The mixture is then cooled to 50° C. and toluene (5 vol) and water (5 vol) are added. The layers are allowed to separate, and the aqueous layer is washed with toluene (10 vol) and then acidified to pH1 with 2.5M HCl solution (7.5 vol). The resultant slurry is filtered and the wet cake is washed with water (2×2 vol). The title product is dried at about 50° C. in a vacuum oven with a nitrogen bleed to constant probe temperature.

Detailed Process Description
1. Added methyl 4-hydroxybenzoate (1 wt, 482.3 g, available from Fluka) to Vessel 1.
2. Added 4-hydroxytetrahydropyran (0.75 vol, 362 mL, 1.2 mole equivalents, available from Sigma-Aldrich) to Vessel 1.
3. Added triphenyl phosphine (2.6 wt, 1253 g, 1.5 mole equivalents) to Vessel 1
4. Purged Vessel 1 with Nitrogen.
5. Added toluene (3.5 vol, 1690 mL) to Vessel 1.
6. Heated contents to 55° C. with stirring.
7. Added diisopropyl azodicarboxylate (DIAD, 1.95 vol, 940 mL, 1.5 mole equivalents, available from Aldrich) to Vessel 1 via a peristaltic pump over 2 hours maintaining the contents temperature at 60±2° C.
8. Stirred contents of Vessel 1 at 60±2° C. for 50 min.
9. Sampled reaction mixture for HPLC analysis.
10. Cooled contents of Vessel 1 to 0-5° C.
11. Seeded batch with triphenylphosphine oxide-diisopropyl hydrazinedicarboxylate adduct (0.001 wt, 0.482 g)
12. Stirred contents of Vessel 1 for 81 min.
13. Filtered off biproduct over 5 min on a PTFE minifilter fitted with Whatman No. 113 wet strengthened filter paper (rough side up). Used 20 L Buchner flask as receiver.
14. Washed wet cake with toluene (2× ca. 1 vol, 2×490 mL) and sucked cake free of solvent.
15. Combined filtrate and cake washes were transferred to Vessel 2 via PTFE suck-up line.
16. Cooled Vessel 2 contents to 0-5° C.
17. Added 2M sodium hydroxide solution (5 vol, 2400 mL) to Vessel 2.
18. Stirred contents of Vessel 2 at 0-5° C. for 5 min before allowing the layers to settle.
19. Ran the lower aqueous layer into a labelled Schott bottle.
20. Added 3M sodium hydroxide solution (5 vol, 2410 mL) to Vessel 2.
21. Heated contents to 80° C., and stirred for 2 hours 45 min.
22. Monitored reaction by HPLC until hydrolysis is complete.
23. Cooled contents of Vessel 2 to 50° C., and then added toluene (5 vol, 2410 mL) to Vessel 2.
24. Added water (5 vol, 2410 mL) to Vessel 2.
25. Stirred contents at 50±5° C. for 5 min before allowing the layers to settle.
26. Ran the lower aqueous layer into a labelled Schott bottle for retention.
27. Ran the upper organic layer into a labelled Schott bottle for disposal.
28. Recharged aqueous layer from labelled Schott bottle to Vessel 2.
29. Added toluene (ca. 10 vol, 4900 mL) to Vessel 2.
30. Stirred contents at 50±5° C. for 5 min before allowing the layers to settle.
31. Ran the lower aqueous layer into a labelled Schott bottle for retention.
32. Ran the upper organic layer into a labelled Schott bottle for disposal.
33. Recharged aqueous layer to Vessel 2.
34. Added 2.5M aqueous hydrochloric acid (7.5 vol, 3620 mL) via peristaltic pump until pH1 is achieved.
35. Stirred the resulting slurry for 15 min.
36. Filtered off product on a PTFE mini filter fitted with Whatman 113 wet strengthened filter paper (rough side up). 10 min filtration time.
37. Washed filter cake with water (2×2vol, 970 mL).
38. Dried the solid product in polythene lined steel trays covered with a muslin cloth, under vacuum and a nitrogen bleed, at 50° C. overnight and at 75° C. for a further 3 days.
39. Title product was obtained as an off-white solid (568.9 g).

Analytical Data

¹H NMR (400 MHz, DMSO-d₆) delta ppm 1.55-1.64 (m, 2H) 1.95-2.03 (m, 2H) 3.49 (ddd, J=11.74, 9.41, 2.57 Hz, 2H) 3.85 (ddd, J=11.80, 4.34, 4.16 Hz, 2H) 4.69 (ddd, J=8.56, 4.65, 4.40 Hz, 1H) 7.03-7.09 (m, 2H) 7.84-7.90 (m, 2H), and 12.31 (br-s, 1H).

In an alternative to the above process, in step 37, the filter cake can be washed with toluene, instead of water, before the 50-75° C. vacuum drying of step 38.

### Drug Preparation Example 1

### (Part A): 1-Isopropyl-4-{[4-(tetrahydro-2H-pyran-4-yloxy)phenyl]carbonyl}hexahydro-1H-1,4-diazepine

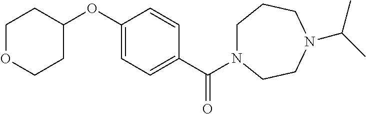

and

### (Parts B and C): 1-Isopropyl-4-{[4-(tetrahydro-2H-pyran-4-yloxy)phenyl]carbonyl}hexahydro-1H-1,4-diazepine hydrochloride

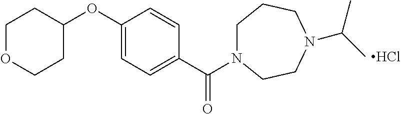

Method

Carbonyl diimidazole (CD) (24 g, 0.8 weight, 1.1 equivalents) was dissolved in acetonitrile (about 300 ml, about 10 volumes) at a contents temperature of about 65° C. (jacket temperature 70° C.) with stirring under nitrogen in a three-necked flask equipped with a thermometer, condenser/nitrogen bubbler, and stopper. Dissolution was complete at about 35-40° C. 4-(Tetrahydro-2H-pyran-4-yloxy)benzoic acid (30 g, 1 weight, 1 equivalent; which can optionally be prepared as described in Drug Preparation Intermediate 2) was added portionwise via the side neck through a solid addition funnel, leading to vigorous gas evolution (CO₂). The addition was completed over 5-10 minutes. The addition funnel was rinsed with acetonitrile (about 45 ml, about 1.5 volumes) which was then added to the reaction mixture. The reaction mixture, activating the acid, was kept at 65° C. (jacket temperature 70° C.) for about 2 hours.

[Here, 1 volume=30 ml; volumes here are with respect to 30 g of input acid.]

Meanwhile, 1-isopropyl-hexahydro-1H-1,4-diazepine bis-trifluoroacetate salt (100 g, e.g. prepared as described in Drug Preparation Intermediate 1) was dissolved in 2M aqueous sodium hydroxide solution (200 ml) and extracted with dichloromethane (2×200 ml). The organic extract was dried (Na₂SO₄) and evaporated to dryness to isolate 1-isopropyl-hexahydro-1H-1,4-diazepine as an oil (27.5 g, 0.916 weight, which if it were pure 1-isopropyl-hexahydro-1H-1,4-diazepine would be 1.43 equivalents).

All this oil was dissolved in acetonitrile (45 ml, 1.5 volumes) and the solution was transferred into the activated acid reaction mixture. The maximum total volume of acetonitrile in the reaction mixture at this stage is 400 ml.

The reaction mixture was conveniently left overnight to react at a contents temperature of 65° C. The reaction mixture was then allowed to cool; then it was clarified and split into three equal parts, Parts A, B and C, each part corresponding to 10 g of input 4-(tetrahydro-2H-pyran-4-yloxy)benzoic acid.

In each of these three Parts A, B and C, 1 volume=10 ml; volumes in these three Parts are with respect to 10 g of input acid (one third of the reaction).

### Drug Preparation Example 1

### Part A: Preparation and isolation of 1-isopropyl-4-{[4-(tetrahydro-2H-pyran-4-yloxy)phenyl]carbonyl}hexahydro-1H-1,4-diazepine (the “free base”)

The solvent was removed from Part A, the first part of the reaction mixture. Isopropyl acetate (10 volumes, 100 ml) was added and the mixture was washed with water (2×3 volumes, 2×30 ml), was dried over Na₂SO₄ and concentrated to 3 volumes (30 ml). Heptane (about 6 volumes, about 60 ml) was added which led to crystallisation of solid, and the mixture was stirred overnight. (That is, the free base was crystallised from about 1:2 isopropyl acetate:heptane.)

The slurry of free base from Part A was filtered and the separated solid was washed with heptane (3 volumes, 30 ml) and dried in a vacuum oven overnight to give 1-isopropyl-4-{[4-(tetrahydro-2H-pyran-4-yloxy)phenyl]carbonyl}hexahydro-1H-1,4-diazepine (the “free base”) (5.24 g).

This sample of free base was suspended in tert-butyl methyl ether (15 volumes with respect to the weight of free base to be recrystallised, about 75 ml) and the mixture was heated to 50° C. whereupon all of the free base dissolved to give a clear solution. Without clarifying it, the solution was allowed to cool gradually to room temperature to give a slurry. The slurry was filtered and the separated solid was dried in a vacuum oven overnight at 50° C. to give 1-isopropyl-4-{[4-(tetrahydro-2H-pyran-4-yloxy)phenyl]carbonyl}hexahydro-1H-1,4-diazepine as a solid (4.15 g).

### Drug Preparation Example 1

### Part B: Preparation and isolation of 1-isopropyl-4-{[4-(tetrahydro-2H-pyran-4-yloxy)phenyl]carbonyl}hexahydro-1H-1,4-diazepine hydrochloride

In parallel, Part B, the second part of the reaction mixture, was concentrated to 2.5 volumes (25 ml). Isopropanol (3 volumes, 30 ml) was added and the mixture was concentrated to 3 volumes (30 ml). Isopropanol (3 volumes, 30 ml) and water (2.0 ml, 0.2 volumes) were added and the resulting mixture (which contained ca. 3.2% water by volume) was heated to 65° C. A solution of HCl in isopropanol (5 to 6 N, 9 ml, 0.9 volumes) was added in one charge. About 10 minutes after the addition was complete, some crystals appeared. The mixture was cooled gradually to 20° C. over 4 hours. At 20° C. the slurry was so thick it would not stir. The slurry was further diluted with 3.2% water in isopropanol; a total of 60 ml (6 volumes) of (3.2% water in isopropanol) was added before a reasonably-stirrable slurry was obtained at 20° C.

The slurry was reheated to 60° C. to obtain a clear solution. This solution was cooled slowly; crystallisation commenced at about 38° C.; the mixture was cooled to 30° C.; and the slurry was stirred overnight at 30° C. The slurry was then cooled to room temperature slowly and was filtered. The resulting solid was washed with isopropanol (4 volumes, 40 ml) and dried in a vacuum oven overnight to give 1-isopropyl-4-{[4-(tetrahydro-2H-pyran-4-yloxy)phenyl]carbonyl}hexahydro-1H-1,4-diazepine hydrochloride as a solid (13.45 g). This can be named the “thermodynamic solid” hydrochloride salt (the result of slow cooling and crystallisation). HPLC suggests that this product contains one impurity present in an amount of ca. 3% (as measured by HPLC peak area). An XRPD analysis indicates that this “thermodynamic solid” hydrochloride salt is crystalline (XRPD has sharp peaks, data not shown).

### Drug Preparation Example 1

### Part C: Preparation and isolation of 1-isopropyl-4-{[4-(tetrahydro-2H-pyran-4-yloxy)phenyl]carbonyl}hexahydro-1H-1,4-diazepine hydrochloride

In parallel, Part C, the third part of the reaction mixture, was concentrated to 2.5 volumes (25 ml). Isopropanol (3 volumes, 30 ml) was added and the mixture was re-concentrated to 3 volumes (30 ml). Isopropanol (7 volumes, 70 ml) and water (4 ml, 0.4 volumes) were added to give a mixture which contained ca. 3.8% water by volume. At room temperature, a solution of HCl in isopropanol (5 to 6 N, 9 ml, 0.9 volumes) was added in one charge, and shortly afterwards crystallisation commenced resulting in a thick slurry which was barely capable of being stirred. This slurry was filtered. The separated solid was washed with isopropanol (4 volumes, 40 ml) and was dried overnight in an oven at 50° C. and under vacuum to give 1-isopropyl-4-{[4-(tetrahydro-2H-pyran-4-yloxy)phenyl]carbonyl}hexahydro-1H-1,4-diazepine hydrochloride as a solid (12.4 g). This can be named the “kinetic solid” hydrochloride salt (the product produced by fast crystallisation). HPLC suggests that this product contains two impurities present in amounts of ca. 2% and ca. 13-14% respectively (as measured by HPLC peak area). XRPD data (not shown) appear to indicate that this “kinetic solid” hydrochloride salt from Part C comprises the same crystalline form as the “thermodynamic solid” hydrochloride salt from Part B.

To improve its purity, this “kinetic solid” hydrochloride salt was recrystallised from acetonitrile and water. The “kinetic solid” hydrochloride salt (11 g) was mixed with acetonitrile (90 ml) but did not completely dissolve in it even at 80° C. Water (1 ml) was added to the stirred slurry and slowly a clear solution was obtained. This stirred solution was allowed to cool to room temperature slowly and was stirred overnight. Filtration and drying gave 1-isopropyl-4-{[4-(tetrahydro-2H-pyran-4-yloxy)phenyl]carbonyl}hexahydro-1H-1,4-diazepine hydrochloride as a solid (9 g). No impurities were seen by HPLC.

### Drug Preparation Example 2

### 1-Isopropyl-4-{[4-(tetrahydro-2H-pyran-4-yloxy)phenyl]carbonyl}hexahydro-1H-1,4-diazepine mono-maleate (crystalline Form 1)

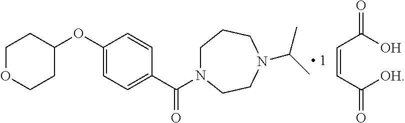

1-Isopropyl-4-{[4-(tetrahydro-2H-pyran-4-yloxy)phenyl]carbonyl}hexahydro-1H-1,4-diazepine (100 mg, prepared as described in Drug Preparation Example 1 Part A, i.e. using the “free base” material which had been recrystallised from tert-butyl methyl ether) was fully dissolved in ethyl acetate (1 ml). Maleic acid (34 mg) was also dissolved in ethyl acetate (1 ml), and the two solutions were combined. On combination, a milky white precipitate was observed, but on stirring a solution was reformed. The solution was left to stand, and after about 1 hour a small amount of white solid had precipitated on the bottom of the vial. This was scratched into the solution, causing the precipitation of a significant quantity of white solid. The very thick slurry was diluted with further ethyl acetate (2 ml) and was subjected to a 0-40° C. temperature cycling program overnight.

After temperature cycling the white solid remained in the mixture. This solid was isolated from the mixture by filtration, and was dried over the weekend in vacuo at 40° C. 1-Isopropyl-4-{[4-(tetrahydro-2H-pyran-4-yloxy)phenyl]carbonyl}hexahydro-1H-1,4-diazepine mono-maleate was obtained as a solid (approx. 93 mg or approx. 95 mg).

A ¹H NMR (nuclear magnetic resonance) spectrum of this Drug Preparation Example 2 product, as a solution in d6-DMSO, appeared to show an approximately stoichiometric quantity (1:1) of maleic acid present with respect to the molar amount of “free base”, and appeared to show no ethyl acetate present. The shift patterns of this solution ¹H NMR spectrum of the mono-maleate prepared in Drug Preparation Example 2 were generally consistent with the shift patterns of the d6-DMSO solution ¹H NMR spectrum of the crystalline Form 1 mono-maleate prepared in Drug Preparation Example 3.

### Drug Preparation Example 2

### Solid-Form Infrared (IR) Data and Spectrum

A solid-form attenuated total reflectance (ATR) infrared (IR) spectrum of the crystalline Form 1 mono-maleate salt prepared in Drug Preparation Example 2 was obtained on a Perkin Elmer Spectrum One FT-IR (Fourier Transform Infrared) spectrometer fitted with a Universal ATR Sampling Accessory (diamond/ZnSe). The sample was prepared by compressing the solid against the ATR cell. The spectrum was recorded with 4 scans at 4 cm⁻¹ resolution.

The solid-form attenuated total reflectance infrared spectrum of the crystalline Form 1 mono-maleate which was obtained comprises inter alia the following bands (peaks) at: 3021, 2958, 2949, 2932, 2864, 2847, 1700, 1622, 1604, 1575, 1509, 1464, 1422, 1393, 1375, 1353, 1341, 1308, 1297, 1280, 1247, 1234, 1205, 1178, 1169, 1153, 1132, 1115, 1089, 1069, 1048, 1017, 1005, 985, 962, 944, 908, 883, 869, 840, 828, 802, 784, 765, 725 and 685 cm⁻¹. It is considered that a likely error in each of the above-mentioned IR bands (peaks) is ±2 cm⁻¹.

In the ATR IR spectrum, relatively more intense IR bands (peaks) were observed at: 1700, 1622, 1464, 1422, 1353, 1247, 1234, 1089, 1048, 869, 840 and 765 cm⁻¹, with a variation for each peak of ±2 cm⁻¹.

### Drug Preparation Example 3

### 1-Isopropyl-4-{[4-(tetrahydro-2H-pyran-4-yloxy)phenyl]carbonyl}hexahydro-1H-1,4-diazepine mono-maleate salt (crystalline Form 1)

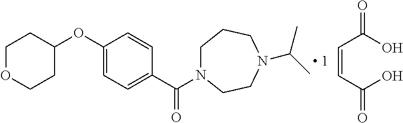

1-Isopropyl-4-{[4-(tetrahydro-2H-pyran-4-yloxy)phenyl]carbonyl}hexahydro-1H-1,4-diazepine (500 mg, 1.44 mmol; preferably prepared as described in Drug Preparation Example 1 Part A that is using the free base material which had been recrystallised from tert-butyl methyl ether) was dissolved in ethyl acetate (5 ml) with stirring and sonication. Maleic acid (169 mg, 1.44 mmol, 1 mole equivalent) was also dissolved in ethyl acetate (5 ml) with sonication. The two solutions were combined dropwise. During the combination a white precipitate began to form, but immediately dissolved on stirring. The solution was then seeded with a few milligrams of a previously-synthesised batch of Form 1 crystalline 1-isopropyl-4-{[4-(tetrahydro-2H-pyran-4-yloxy)phenyl]carbonyl}hexahydro-1H-1,4-diazepine mono-maleate (prepared according to Drug Preparation Example 2). The seed took, precipitation was seen, and over the next few minutes the slurry thickened. Further ethyl acetate (3 ml) was added, and the slurry was left to stir for about 60 minutes before being transferred to a 0-40° C. temperature cycling program and left for 3 nights (over the weekend).

After temperature cycling the thick slurry remained, and the resulting solid was isolated by filtration, and dried overnight in vacuo at 40° C. 1-Isopropyl-4-{[4-(tetrahydro-2H-pyran-4-yloxy)phenyl]carbonyl}hexahydro-1H-1,4-diazepine mono-maleate was obtained as a white solid (592 mg, 88% theoretical yield).

Samples of this material, Drug Preparation Example 3, were removed for a range of analyses, including X-ray powder diffraction (XRPD) (see Table 1 below) and solid-form infrared (IR) spectroscopy (data not shown, but giving a solid-form IR spectrum consistent with that of the Form 1 crystalline mono-maleate produced in Drug Preparation Example 2). Also, a ¹H NMR (nuclear magnetic resonance) spectrum in d6-DMSO solution was run—see below for analytical details.

Inter alia from the XRPD diffractogram (not shown), which has sharp peaks, it appears that the solid mono-maleate salt is crystalline. This crystalline form of 1-isopropyl-4-{[4-(tetrahydro-2H-pyran-4-yloxy)phenyl]carbonyl}hexahydro-1H-1,4-diazepine mono-maleate is named as crystalline Form 1.

Gravimetric vapour sorption (GVS) analysis, which measures water sorption/desorption at different relative humidities, showed the sample of the Form 1 crystalline mono-maleate salt, prepared in Drug Preparation Example 3, reversibly adsorbed and/or absorbed, and/or desorbed, approximately 0.4% w/w water across the 0-90% relative humidity range at 25° C. under nitrogen. This appears to represent an advantage, with respect to drug developability, over the solid hydrochloride salt of Drug Preparation Example 1 Part C (the “kinetic” solid before purification by recrystallisation), which adsorbed and/or absorbed, and/or desorbed, higher quantities of water over the same relative humidity range (data not shown).

### X-Ray Powder Diffraction (XRPD) Data and Diffractogram for Crystalline Form 1 of 1-isopropyl-4-{[4-(tetrahydro-2H-pyran-4-yloxy)phenyl]carbonyl}hexahydro-1H-1,4-diazepine Mono-Maleate from Drug Preparation Example 3

X-ray powder diffraction (XRPD) data, on the crystalline Form 1 of 1-isopropyl-4-{[4-(tetrahydro-2H-pyran-4-yloxy)phenyl]carbonyl}hexahydro-1H-1,4-diazepine mono-maleate prepared in Drug Preparation Example 3, were acquired on a Phillips PANalytical X′Pert Pro powder diffractometer model PW3040/60, serial number DY1850, equipped with an X′Celerator detector (available from PANalytical UK, 7310 IQ Cambridge, Waterbeach, Cambridge CB25 9AY, United Kingdom). The acquisition conditions were: radiation: Cu Kα (copper K-alpha), generator tension: 40 kV, generator current: 45 mA, start angle: 2.0° 2θ, end angle: 40.0° 2θ, step size: 0.0167° 2θ. The time per step was 31.750 s. The sample was prepared by mounting a few milligrams of sample on a Si (silicon) wafer (zero background) plate, resulting in a thin layer of powder. The XRPD data were collected at room temperature and at atmospheric pressure in air.

Potentially characteristic peak positions, in degrees two-theta (2θ), and calculated d-spacings, in Angstroms (Å), are summarised in Table 1 below. These were calculated from the raw data using Highscore software (available from PANalytical UK, 7310 IQ Cambridge, Waterbeach, Cambridge CB25 9AY, United Kingdom). Experimental error in the peak positions is approximately ±0.1° two-theta (2θ). Relative peak intensities will vary due to preferred orientation.

### Table 1—XRPD Data for Crystalline Form 1 Mono-Maleate from Drug Preparation Example 3

| 2θ/° | d-spacing/Å |
|---|---|
| 9.2 | 9.6 |
| 11.8 | 7.5 |
| 13.4 | 6.6 |
| 14.7 | 6.0 |
| 15.8 | 5.6 |
| 16.2 | 5.5 |
| 17.0 | 5.2 |
| 18.5 | 4.8 |
| 19.8 | 4.5 |
| 20.3 | 4.4 |
| 20.5 | 4.3 |
| 21.0 | 4.2 |
| 21.3 | 4.2 |
| 27.1 | 3.3 |
| 27.4 | 3.3 |
| 27.8 | 3.2 |
| 28.4 | 3.1 |
| 30.2 | 3.0 |

Of the peaks listed in Table 1, seven XRPD peaks, which are of medium or strong intensity and/or which are believed most likely to be seen in a sample of test material containing the crystalline Form 1 mono-maleate, and some or all of which are more likely to be characteristic of the crystalline Form 1, are observed at the following degrees two-theta (2θ) values: 9.2±0.1°, 13.4±0.1°, 17.0±0.1°, 18.5±0.1°, 19.8±0.1°, 21.3±0.1°, and 27.8±0.1°.

### ¹H NMR (Nuclear Magnetic Resonance) Solution Spectrum of Crystalline Form 1 Mono-Maleate from Drug Preparation Example 3

A ¹H NMR (nuclear magnetic resonance) spectrum of the crystalline Form 1 of 1-isopropyl-4-{[4-(tetrahydro-2H-pyran-4-yloxy)phenyl]carbonyl}hexahydro-1H-1,4-diazepine mono-maleate prepared in Drug Preparation Example 3, was obtained as a solution in d6-DMSO (d6-dimethyl sulfoxide) using a 400 MHz ¹H NMR spectrometer. The ¹H NMR spectrum, in which only 0 to 10 ppm is reported, has peaks at delta (ppm) as follows (integrals/numbers of hydrogens (H) are given in brackets, s=singlet, d=doublet, m=multiplet): 9.03 (0.78H, broad s), 7.41 (2.00H, d), 7.05 (2.00H, d), 6.04 (1.96H, s), 4.65 (1.01H, m), 4.01 (about 0.7H, broad m), 3.87 (about 2.6H, m), 3.1 to 3.8 (about 18.2H, m, includes triplet at 3.51 ppm and strong peak at 3.32 ppm), 2.52 (20.0H, s), 2.00 (4.03H, broad d), 1.61 (2.07H, m), 1.26 (6.13H, s or m). These reported peaks include the peak due to incompletely-deuterated DMSO.

The solution ¹H NMR spectrum of the Drug Preparation Example 3 mono-maleate reported above:

(i) appears to show some shifting of peaks of the active drug compound (cf. those of the “free base”) indicating that salt formation is likely to have occurred,

(ii) appears to show that the maleic acid had also been shifted (cf. free maleic acid itself),

(iii) appears to show that an approximately stoichiometric quantity (1:1) of maleic acid is present with respect to the molar amount of “free base”, and

(iv) appears to show no ethyl acetate present.

The shift patterns of this d6-DMSO solution ¹H NMR spectrum of the crystalline Form 1 mono-maleate prepared in Drug Preparation Example 3 were generally consistent with the shift patterns of the d6-DMSO solution ¹H NMR spectrum of the crystalline Form 1 mono-maleate prepared in Drug Preparation Example 2 (data not shown).

### DOSAGE FORM EXAMPLES

### Example 1

### Preparation of Round Tablets Containing 0.01mg 1-isopropyl-4-{[4-(tetrahydro-2H-pyran-4-yloxy)phenyl]carbonyl}hexahydro-1H-1,4-diazepine or a Pharmaceutically Acceptable Salt Thereof

The core components are passed through a nominal 30 mesh screen and then blended together in a suitable blender and compressed on a rotary tablet press to produce round biconcave tablets with a diameter of 7.9 mm and an approximately 0.8 mm deep trough on both sides of the tablet. Compression is followed by de-dusting and metal checking. The tablets are then transferred to a coating pan and coated to a target 4% (w/w) gain.

The composition of the carrier tablets is given below:

Core

| | | % w/w of |
|---|---|---|
| | Weight | uncoated |
| Component | (mg per tablet) | carrier tablet |
| Microcrystalline cellulose (Avicel ™ PH- | 162 mg | 90% w/w |
| 102) | | |
| Pregelatinized Starch (Starch 1500) | 16.2 mg | 9% w/w |
| Magnesium Stearate | 1.8 mg | 1% w/w |
| Total carrier tablet (uncoated) | 180 mg | |

Film Coat

| | | % w/w of | % w/w of |
|---|---|---|---|
| | | coated | uncoated |
| | Weight | carrier | carrier |
| Component | (mg per tablet) | tablet | tablet |
| Opadry White YS-1-7003 ™ | 7.2 mg | 3.85% w/w | 4% w/w |
| Purified Water | qs* | | |
| Total carrier tablet (coated) | 187.2 mg | | |

*Removed during processing

A carrier solution is prepared by dissolving 5 g hydroxypropyl cellulose (HPC) (Klucel™ Grade EF, available from Aqualon), and 3 g anhydrous citric acid in methanol, filtering through a 10 micron filter and then bringing the final volume to 100 ml with methanol.

1-Isopropyl-4-{[4-(tetrahydro-2H-pyran-4-yloxy)phenyl]carbonyl}hexahydro-1H-1,4-diazepine or a pharmaceutically acceptable salt thereof is dissolved in the carrier solution with a sonicator (and by also using a magnetic stirrer) until a uniform solution is obtained with a final concentration of 2.5 mg/g (w/w) (measured as the free base).

4 mg of carrier solution is dispensed onto each tablet in an array of carrier tablets. The tablets are dried in a forced air oven at about 50° C. for 10-20 minutes.

The composition of the finished tablets is as follows:

| | Weight | | % w/w of |
|---|---|---|---|
| Component | (mg per tablet) | | total tablet |
| 1-isopropyl-4-{[4-(tetrahydro-2H-pyran-4- | 0.01 | mg | ca. 0.0053% |
| yloxy)phenyl]carbonyl}hexahydro-1H-1,4- | | | |
| diazepine or a pharmaceutically acceptable | | | |
| salt thereof (measured as the free base) | | | |
| Carrier (5% HPC/3% Citric acid in | 0.4 | mg | ca. 0.213% |
| methanol*), containing: | | | |
| hydroxypropylcellulose | ca. 0.25 | mg | ca. 0.133% |
| citric acid | ca. 0.15 | mg | ca. 0.080% |
| Carrier tablet (coated) | 187.2 | mg | 99.78 to |
| | | | 99.79% |
| Total tablet | 187.6 | | |

*the methanol is removed during manufacturing process

### Example 1A

In an alternative embodiment of Example 1, solid (e.g. crystalline) 1-isopropyl-4-{[4-(tetrahydro-2H-pyran-4-yloxy)phenyl]carbonyl}hexahydro-1H-1,4-diazepine mono-maleate is dissolved in the carrier solution (the HPC and citric acid in methanol). More particularly crystalline Form 1 of 1-isopropyl-4-{[4-(tetrahydro-2H-pyran-4-yloxy)phenyl]carbonyl}hexahydro-1H-1,4-diazepine mono-maleate is used as a starting material. Thus a tablet is prepared comprising 1-isopropyl-4-{[4-(tetrahydro-2H-pyran-4-yloxy)phenyl]carbonyl}hexahydro-1H-1,4-diazepine mono-maleate (measured as the free base), HPC and citric acid in a film, wherein the film is present in a recess (trough) on one side of the carrier tablet.

### Examples 2, 3 and 4

### Preparation of Round Tablets Containing 0.02 mg, 0.05 mg or 0.5 mg 1-isopropyl-4-{[4-(tetrahydro-2H-pyran-4-yloxy)phenyl]carbonyl}hexahydro-1H-1,4-diazepine or a Pharmaceutically Acceptable Salt Thereof

Tablets containing 0.02 mg, 0.05 mg or 0.5 mg 1-isopropyl-4-{[4-(tetrahydro-2H-pyran-4-yloxy)phenyl]carbonyl}hexahydro-1H-1,4-diazepine or a pharmaceutically acceptable salt thereof (measured as the free base) can be prepared in the manner described in Example 1 except that the concentration of 1-isopropyl-4-{[4-(tetrahydro-2H-pyran-4-yloxy)phenyl]carbonyl}hexahydro-1H-1,4-diazepine or a pharmaceutically acceptable salt thereof in the carrier solution is varied.

### Examples 2A, 3A and 4A

In an alternative embodiment of Examples 2, 3 and 4, solid (e.g. crystalline) 1-isopropyl-4-{[4-(tetrahydro-2H-pyran-4-yloxy)phenyl]carbonyl}hexahydro-1H-1,4-diazepine mono-maleate is dissolved in the carrier solution (the HPC and citric acid in methanol). More particularly crystalline Form 1 of 1-isopropyl-4-{[4-(tetrahydro-2H-pyran-4-yloxy)phenyl]carbonyl}hexahydro-1H-1,4-diazepine mono-maleate is used as a starting material. Thus a tablet is prepared comprising 1-isopropyl-4-{[4-(tetrahydro-2H-pyran-4-yloxy)phenyl]carbonyl}hexahydro-1H-1,4-diazepine mono-maleate (measured as the free base), HPC and citric acid in a film, wherein the film is present in a recess (trough) on one side of the carrier tablet.

### Example 5

### Further Coating, with a Pad Printed Overcoat, of the Tablets Prepared in Any of Examples 1, 2, 3, 4, 1A, 2A, 3A and 4A

The tablets prepared in any of Examples 1, 2, 3, 4, 1A, 2A, 3A and 4A can optionally be further coated so that they comprise a further coating over (covering) all of the film which contains the active compound or salt. This optional further coating is a pad printed overcoat, i.e. is an overcoat which is prepared by a pad printing process.

The pad printed overcoat comprises titanium dioxide and Nisso™ HPC (hydroxypropylcellulose) Grade SSL. The pad printing overcoat process uses one of the following coating mixtures comprising HPC and titanium dioxide in ethanol, all of which have the following generalised composition: 20 to 25% Nisso™ HPC SSL; 31 to 38% titanium dioxide; and 41 to 49% ethanol:

### Example 5A

### 22% Nisso™ HPC SSL; 34% Titanium Dioxide; 44% Ethanol

### Example 5B

### 21% Nisso™ HPC SSL; 38% Titanium Dioxide; 41% Ethanol

### Example 5C

### 25% Nisso™ HPC SSL; 34% Titanium Dioxide; 41% Ethanol

### Example 5D

### 20% Nisso™ HPC SSL; 31% Titanium Dioxide; 49% Ethanol

After production, the pad printed overcoat on the tablets can then optionally be further printed with any desired design.

The following examples (Example 6 and Example 7) are representative of an example tablet which may be prepared in accordance with the invention:

### Example 6

### Preparation of a Orally Disintegrating Tablet (ODT) Carrier Substrate

a) Preparation of ODT Carrier Substrate

StarLac and Neotame are passed through a nominal 20 mesh screen. The mixture and unsieved mint flavoring are transferred to a suitable blender and blended for approximately 10 minutes. Magnesium stearate is passed through a nominal 30 mesh screen, transferred to the blender and the entire mixture blended for approximately 2 minutes. The weights of material used are calculated from the percentage weights given in Table A. The blend is compressed to meet the desired specifications (for example round, biconcave tablets, range in diameter from ˜8 mm to ˜9.5 mm) on a suitable rotary press utilizing appropriate tablet tooling. The tablets are passed through a de-duster and metal checker.

TABLE A

| COMPOSITION | SPECIFICATION | % (w/w) | FUNCTION |
|---|---|---|---|
| StarLac* | Non Compendial | 98.5% | Diluent |
| Mint Flavoring | Non Compendial | 0.9% | Flavoring |
| Neotame | NF | 0.1% | Sweetener |
| Magnesium Stearate | Ph. Eur/USP-NF/JP | 0.5% | Lubricant |

*StarLac: mixture of 85% alpha-lactose monohydrate (Ph. Eur./USP-NF) and 15% maize starch (Ph. Eur./USP-NF)

b) Preparation of Ethylcellulose Coat for Application to Tablet by Pad-Printing

Ethylcellulose is dissolved in methanol with stirring, and triethyl citrate added. The weights of material used are calculated from the percentage weights given in Table B. Sufficient methanol is added to bring to target on w/w basis. The solution is transferred to the ink cup of a pad printing machine equipped with a suitable image cliché with a round image, slightly smaller diameter then the actual tablet diameter. A suitable polymer pad is installed to match the cliché image plate. Tablets are presented to the pad printer in a defined array, matching the cliché. The pad printer may apply 2-4 tamps to the carrier tablet to apply a coat that will provide a protective layer to mitigate solvent infiltration into the uncoated carrier substrate during the liquid dispensing process.

TABLE B

| COMPOSITION | SPECIFICATION | % (w/w) | FUNCTION |
|---|---|---|---|
| Ethylcellulose | NF | 30 | Protective |
| | | | barrier coat |
| Triethyl Citrate | Ph. Eur./USP-NF | 1.67 | Plasticiser |
| Methanol | Ph. Eur./USP-NF | qs*** to 100 | vehicle |

***Methanol eliminated by evaporation.

### Example 7

### Alternative Preparation of an Orally Disintegrating Tablet (ODT) Carrier Substrate

Mannitol, crospovidone XL, xylitol and Neotame are passed through a nominal 20 mesh screen, the mixture and the unsieved Mint Flavoring transferred to a suitable blender and blended for approximately 10 minutes. Magnesium stearate and colloidal silicon dioxide are passed through a nominal 30 mesh screen, transferred to the blender and the entire mixture blended for approximately 2 minutes. The weights of material used are calculated from the percentage weights given in Table C. The blend is compressed to meet the desired specifications (for example round, biconcave tablets, range in diameter from ˜8 mm to ˜9.5 mm) on a suitable rotary press utilizing an appropriate tablet tooling. The tablets are passed through a de-duster and metal checker.

An ethylcellulose coat may be prepared and applied as described for Example 6.

TABLE C

| COMPOSITION | SPECIFICATION | % (w/w) | FUNCTION |
|---|---|---|---|
| Mannitol (Grade 300 | Ph. Eur/USP-NF/JP | 73.15% | Diluent/ |
| Direct Compression) | | | sweetener |
| Crospovidone XL | Ph. Eur/USP-NF | 20.00% | Disintegrant |
| Xylitol (Grade 300 | Ph. Eur/USP-NF/JP | 5.00% | Diluent/ |
| for Direct | | | sweetener |
| Compression) | | | |
| Mint Flavoring | Non Compendial | 0.90% | Flavoring |
| Neotame | NF | 0.10% | Sweetener |
| Magnesium Stearate | Ph. Eur/USP-NF/JP | 0.75% | Lubricant |
| Colloidal silicon | Ph. Eur/USP-NF/JP | 0.10% | Lubricant |
| dioxide | | | |

PROPERTIES: The stability of the drug substance in the tablets may be tested as set out below:

Dissolve 5 tablets in diluent (1:9 acetonitrile: 50 mM potassium dihydrogen orthophosphate, adjusted to pH 3 with orthophosphoric acid) to produce a final concentration of active agent of between 1-10 μg/ml and sonicate for 10 minutes. Check for complete disintegration and sonicate further if required. Allow to cool to ambient temperature and then centrifuge an aliquot of the sample at 14,000 rpm. Prepare samples using placebo tablets to act as control samples.

Using the following instrument conditions, equilibrate the chromatographic system with 95% A, 5% B. Record the chromatograms for the sample and placebo preparations.

Column: XBridge C18 3 μM 15 cm×4.6 mm i.d.

Column Temperature: 40° C.

Mobile Phase A: 10 mM ammonium bicarbonate, pH 10 with ammonia

Mobile Phase B: Acetonitrile

Flow Rate: 1 ml/min

Detector wavelength: 250 nm

Injection volume: 100 μl

Gradient Profile:

| Time (min) | % A | % B |
|---|---|---|
| 0 | 95 | 5 |
| 5 | 95 | 5 |
| 30 | 30 | 70 |
| 30.1 | 95 | 5 |

Following comparison of the chromatograms to identify impurities/degradation products, the percentage content of each impurity/degradation product in the control and sample injections can be calculated by dividing the area of the impurity/degradation product peak by the summed total of the peak for 1-isopropyl-4-{[4-(tetrahydro-2H-pyran-4-yloxy)phenyl]carbonyl}hexahydro-1H-1,4-diazepine or a pharmaceutically acceptable salt thereof and all impurities/degradation products, and multiplying by 100.

The total impurity/degradation product content can be calculated by summing the percentage content of each impurity/degradation product present. Typically, only impurities/degradation products present in an amount of greater than or equal to 0.05 or 0.03% are included in the calculation of total impurity/degradation product content.

1-30. (canceled)

31. A dosage form for oral administration comprising a carrier tablet, wherein the carrier tablet is at least partially covered by a film comprising 1-isopropyl-4-{[4-(tetrahydro-2H-pyran-4-yloxy)phenyl]carbonyl}hexahydro-1H-1,4-diazepine 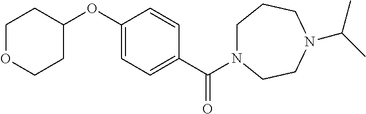
or a pharmaceutically acceptable salt thereof.

32. The dosage form as claimed in claim 31, comprising:
a) the 1-isopropyl-4-{[4-(tetrahydro-2H-pyran-4-yloxy)phenyl]carbonyl}hexahydro-1H-1,4-diazepine 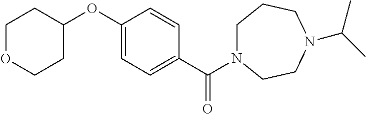
or the pharmaceutically acceptable salt thereof;
b) optionally, a stabiliser, which reduces degradation of the 1-isopropyl-4-{[4-(tetrahydro-2H-pyran-4-yloxy)phenyl]carbonyl}hexahydro-1H-1,4-diazepine or the pharmaceutically acceptable salt thereof in the dosage form when compared to a dosage form lacking said stabiliser; and
c) a pharmaceutically acceptable excipient.

33. The dosage form as claimed in claim 31, wherein the film, which at least partially covers the carrier tablet, comprises a stabiliser that reduces degradation of the 1-isopropyl-4-{[4-(tetrahydro-2H-pyran-4-yloxy)phenyl]carbonyl}hexahydro-1H-1,4-diazepine or the pharmaceutically acceptable salt thereof in the dosage form, when compared to a dosage form lacking said stabiliser, wherein said stabiliser comprises citric acid.

34. The dosage form as claimed in claim 33, wherein the molar ratio of the 1-isopropyl-4-{[4-(tetrahydro-2H-pyran-4-yloxy)phenyl]carbonyl}hexahydro-1H-1,4-diazepine or the pharmaceutically acceptable salt thereof, measured as the free base, to the citric acid is in the range of 1.5:1 to 1:500.

35. The dosage form as claimed in claim 31, wherein the film, which at least partially covers the carrier tablet, additionally contains a film former.

36. The dosage form as claimed in claim 35, wherein the film former is hydroxypropylcellulose.

37. The dosage form as claimed in claim 31, wherein the carrier tablet comprises a diluent or a mixture of diluents, present at 70-100% by weight of the carrier tablet, wherein the diluent includes lactose, sucrose, dextrose, mannitol, sorbitol, starch, microcrystalline cellulose, calcium sulphate and/or dibasic calcium phosphate (CaHPO₄).

38. The dosage form as claimed in claim 31, which contains from 10 μg to 2 mg of the 1-isopropyl-4-{[4-(tetrahydro-2H-pyran-4-yloxy)phenyl]carbonyl}hexahydro-1H-1,4-diazepine or the pharmaceutically acceptable salt thereof, when measured as the amount of free base present.

39. The dosage form as claimed in claim 31, wherein the film contains 1-isopropyl-4-{[4-(tetrahydro-2H-pyran-4-yloxy)phenyl]carbonyl}hexahydro-1H-1,4-diazepine mono-maleate.

40. The dosage form as claimed in claim 38, wherein the film contains crystalline Form 1 of 1-isopropyl-4-{[4-(tetrahydro-2H-pyran-4-yloxy)phenyl]carbonyl}hexahydro-1H-1,4-diazepine mono-maleate which is characterized by having:
(a) an X-ray powder diffraction diffractogram comprising four or more of the following peaks at substantially the following degrees two-theta values:
9.2±0.1°, 13.4±0.1°, 17.0±0.1°, 18.5±0.1°, 19.8±0.1°, 21.3±0.1°, and 27.8±0.1°;
wherein the X-ray powder diffraction diffractogram is measured with a X-ray powder diffractometer using copper K-alpha X-radiation and a step size of 0.0167° two-theta or less; and/or
(b) a solid-form attenuated total reflectance infrared spectrum comprising five or more of the following peaks:
1700, 1622, 1464, 1422, 1353, 1247, 1234, 1089, 1048, 869, 840 and 765 cm−1;
with a variation allowed for each peak of ±2 cm−1.

41. The dosage form as claimed in claim 31, wherein said carrier tablet has at least one recess, wherein said film is present in a recess on said carrier tablet.

42. The dosage form as claimed in claim 40, wherein said carrier tablet is coated.

43. The dosage form as claimed in claim 31, wherein said dosage form is further coated.

44. A method of producing the dosage form as defined in claim 31, comprising dispensing a solution or suspension of 1-isopropyl-4-{[4-(tetrahydro-2H-pyran-4-yloxy)phenyl]carbonyl}hexahydro-1H-1,4-diazepine or a pharmaceutically acceptable salt thereof, and optionally a stabiliser, onto a carrier tablet, optionally wherein the carrier tablet and the dispensed solution or suspension is heated to evaporate excessive liquid and to result in the formation of a film upon at least a part of the surface of the carrier tablet.

45. A method as claimed in claim 43, wherein a film former being hydroxypropylcellulose is present in the solution or suspension in an amount between 4-6% w/v.

46. A method of treatment of cognitive impairment in Alzheimer's disease, cognitive impairment in schizophrenia, attention deficit hyperactivity disorder, age-related memory dysfunction, mild cognitive impairment, or Parkinson's disease in a human in need thereof, which comprises administering to said human the dosage form as defined in claim 31.
